(19) United States
(12) Patent Application Publication
(10) Pub. No.: US 2025/0152252 A1
(43) Pub. Date: May 15, 2025

(54) MRI BASED NAVIGATION

(71) Applicant: Covidien LP, Mansfield, MA (US)

(72) Inventors: Oren P. Weingarten, Hod-Hasharon (IL); Scott E.M. Frushour, Boulder, CO (US); John W. Komp, Dillon, CO (US); Scott Robert Tiesma, Brooklyn Park, MN (US); Evgeni Kopel, Barkan (IL)

(73) Assignee: Covidien LP, Mansfield, MA (US)

(21) Appl. No.: 18/840,048

(22) PCT Filed: Feb. 17, 2023
(86) PCT No.: PCT/IB2023/051468

§ 371 Date: Aug. 20, 2024

Related U.S. Application Data
(60) Provisional application No. 63/312,898, filed on Feb. 23, 2022.

(51) Int. Cl. A61B 34/20; A61B 34/10

(52) U.S. Cl. CPC A61B 34/20; A61B 34/10; A61B 2034/105; A61B 2034/2048; A61B 2034/2065

(57) ABSTRACT

A system and method for luminal navigation of a catheter including a sensor and a computing device. The computing device executing steps of receiving magnetic resonance signals from a magnetic resonance image (MRI) scanner and to generate an MRI image data set, generating a three-dimensional (3D) model from the MRI image data set, generating a pathway through the 3D model to a target, determining a location of the sensor within the patient, displaying a location of a portion of the catheter in the 3D model, updating the displayed location of the portion of the catheter, receiving second magnetic resonance signals and generate a second MRI image data set, receiving an indication of a distal end of the catheter in the second MRI image data set, and updating a relative position of a distal end of the catheter and the target in the 3D model.

### FIELD

The disclosure relates to surgical imaging systems, and more particularly, to systems and methods for assisting a clinician in navigation of catheters and other tools to specific locations within a patient for biopsy and therapy while reducing the ionizing radiation exposure of the patient and surgical team.

### BACKGROUND

There exist several commonly applied medical methods, such as endoscopic procedures or minimally invasive procedures, for diagnosing or treating various maladies affecting organs including the liver, brain, heart, lung, gall bladder, kidney and bones. Often, one or more imaging modalities, such as computed tomography (CT), fluoroscopy, and other x-ray radiation-based imaging technique is employed by clinicians to identify and navigate to areas of interest within a patient and ultimately a specific location (e.g., a target for biopsy or treatment).

For example, an endoscopic approach has proven useful in navigating to areas of interest within a patient, and particularly so for areas within luminal networks of the body such as the lungs. To enable the endoscopic approach, and more particularly the bronchoscopic approach in the lungs, endobronchial navigation systems have been developed that use pre-procedural or previously acquired CT image data to generate a three-dimensional (3D) renderings or models of the particular body part. Additional developments have enabled the use of fluoroscopic images, which can be generated intra-procedurally and at much lower cost. Whether using CT image data or fluoroscopic image data the resulting 3D model or rendering generated from the image data is then utilized to create or modify a navigation plan to facilitate the advancement of a navigation catheter (or other suitable medical device) either alone or through the bronchoscope or a guide sheath and the luminal network, for example the airways of a patient's lungs to an identified target or area of interest.

To be of use in navigation to a target or area of interest within the patient's lungs the 3D model or rendering of the lungs derived from the pre-procedural images must be registered to the patient's lungs. After an initial registration and following navigation proximate to the previously identified target it is often necessary to capture additional intra-procedural image data (e.g., CT image data, cone-beam CT image data, or fluoroscopic image data) in order to correct for CT-to-body divergence (i.e., conduct a local registration). The purpose of the local registration is to determine the in situ relative positions of the navigation catheter and the target and to update of their relative positions in the 3D model formed from the pre-procedure imaging. Following further navigation and placement of the biopsy or therapy tool in the target, yet further intra-procedural imaging may be acquired to confirm placement in the target.

All of these image data sets being acquired of the patient increase the total ionizing radiation dose received by the patient. Similarly, as multiple of these image data sets are acquired intra-procedurally, the medical team (pulmonologists, surgeons, nurses, etc.) receive multiple doses of ionizing radiation per procedure and thus may receive significant and damaging exposure as they examine and treat many patients over the course of their careers.

Thus, while the current systems are very useful for their intended purpose and have been employed to diagnose and treat many people, the systems can always use improvements.

### SUMMARY

One aspect of the disclosure is directed to a system for luminal navigation. The system includes a catheter configured for navigation within a luminal network of a patient, a sensor; and a computing device including a processor and computer readable memory, the memory storing thereon instructions that when executed by the processor to: receive magnetic resonance signals from a magnetic resonance image (MRI) scanner and to generate an MRI image data set; generate a three-dimensional (3D) model from the MRI image data set; generate a pathway through the 3D model to a target; determine a location of the sensor within the patient; cause display of a location of a portion of the catheter in the 3D model; and update the displayed location of the portion of the catheter; receive second magnetic resonance signals and generate a second MRI image data set; receive an indication of a distal end of the catheter in the second MRI image data set; and update a relative position of a distal end of the catheter and the target in the 3D model. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The system where the instructions when executed by the processor cause the display of the updated relative position of the distal end of the catheter and the target in the 3D model. The instructions when executed by the processor receive third magnetic resonance signals to form a third MRI image to confirm placement of the catheter, a biopsy tool, or a therapy tool in the target. The instructions when executed by the processor determine whether more targets exist in the 3D model. The instructions when executed by the processor cause the display of the 3D model and a pathway to a second target. The system further including a magnetic resonance scanner generating the magnetic resonance signals. The instructions when executed by the processor cause the generation of an electromagnetic field and the sensor is an electromagnetic sensor. The system further including a transmitter mat generating the electromagnetic field. A magnetic coil of the MRI scanner generates the electromagnetic field. The sensor is an inertial measurement unit. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A further aspect of the disclosure is directed to a method of navigating a catheter to a target within a patient. The method of navigating also includes receiving magnetic resonance signals from a magnetic resonance image (MRI) scanner and generating an MRI image data set; generating a three-dimensional (3D) model from the MRI image data set, generating a pathway through the 3D model to a target, determining a location of a sensor within the patient, causing display of a location of a portion of a catheter in the 3D model based on the determined position of the sensor, and updating a displayed location of the portion of the catheter, receiving second magnetic resonance signals and generate a second MRI image data set, receiving an indication of a distal end of the catheter in the second MRI image data set, and updating a relative position of a distal end of the catheter and the target in the 3D model. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method further including causing display of the updated relative position of the distal end of the catheter and the target in the 3D model. The method further including receiving third magnetic resonance signals to form a third MRI image to confirm placement of the catheter, a biopsy tool, or a therapy tool in the target. The method further including generating an electromagnetic field and the determining a location of the sensor in the electromagnetic field. The electromagnetic field is generated by a transmitter mat. A magnetic coil of the MRI scanner generates the electromagnetic field. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Yet a further aspect of the disclosure is directed to a method of navigating a catheter to a target within a patient. The method of navigating also includes receiving magnetic resonance signals from a magnetic resonance image (MRI) scanner and generating an MRI image data set; generating a three-dimensional (3D) model from the MRI image data set; generating a pathway through the 3D model to a target; determining a location of a distal portion of a catheter within the 3D model; causing display of the location of at least the distal portion of a catheter in the 3D model; and receiving signals from a sensor incorporated in the catheter; updating a displayed location of at least a portion of the catheter based on the received signals; receiving second magnetic resonance signals and generate a second MRI image data set, where the second MRI image data set is focused to an area proximate the sensor; and updating a displayed position of the distal portion of the catheter in the 3D model based on the second MRI image data set. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method where the sensor is an inertial measurement unit (IMU). The method where the updated displayed position of the distal portion of the catheter is employed to eliminate drift of the IMU. The method where the additional MRI image data sets are focused to the area proximate the sensor; and updating the displayed position of the distal portion of the catheter in the 3D model based on the second MRI image data set and employing the updated displayed position of the catheter to eliminate drift of the IMU. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

Various aspects and features of the disclosure are described hereinbelow with references to the drawings, wherein:

FIG. 1 depicts a schematic of an imaging and luminal network navigation system in accordance with the disclosure;

FIG. 2 is a perspective view of an imaging and luminal network navigation system in accordance with the disclosure

FIG. 3 is a perspective view of a bedside magnetic resonance imaging device in accordance with the disclosure; and

FIG. 4 is a flowchart of a method in accordance with the disclosure.

### DETAILED DESCRIPTION

The disclosure relates to surgical imaging systems, and more particularly, to systems and methods for assisting a clinician in navigation of catheters and tools to targets within a patient for biopsy and therapy while reducing the ionizing radiation exposure of the patient and surgical team. To significantly reduce the ionizing radiation exposure of the patient and medical staff, rather than employing intra-procedural fluoroscopy, CT, or cone-beam CT image data a non-radiating imaging modality such as magnetic resonance imaging (MRI). The ionizing radiation exposure of the patient can be further reduced by employing no pre-procedural imaging, and instead utilizing an intra-procedural MRI image data set for the purpose of identifying targets to navigate to and to generate the 3D model.

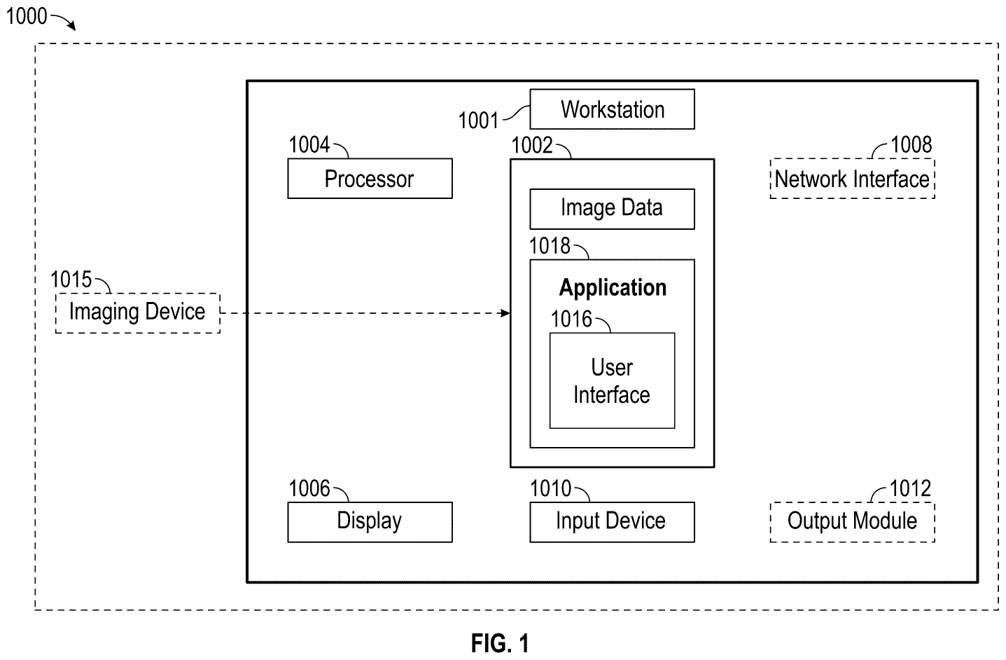

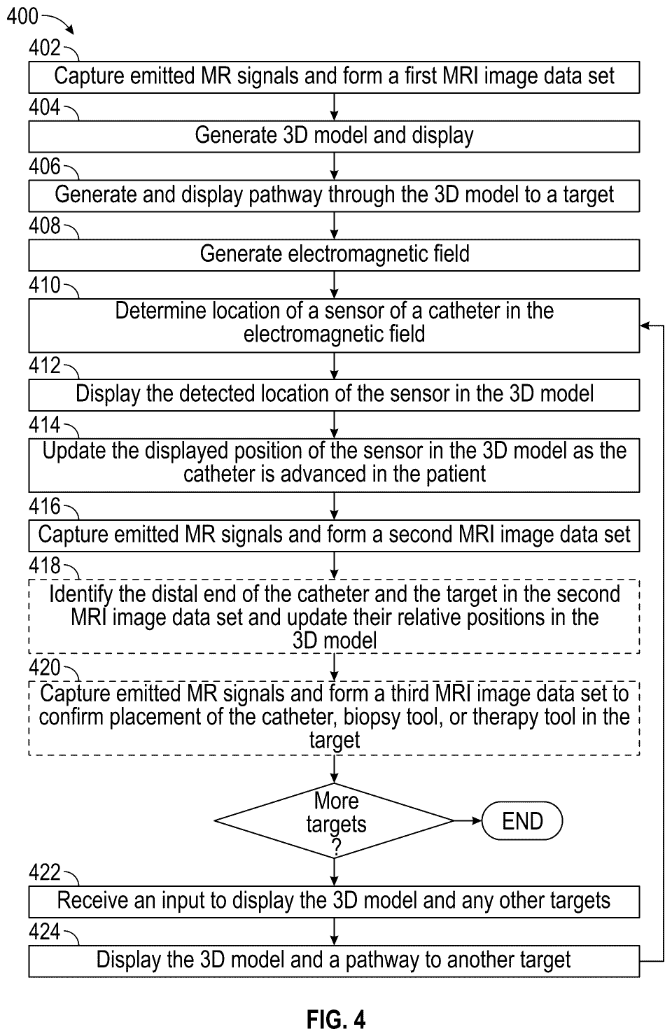

Reference is now made to FIG. 1, which is a schematic diagram of a system 1000 configured for use with the methods of the disclosure including the methods of FIG. 4 System 1000 may include a workstation 1001, and optionally an imaging device such as a bedside imaging device 1015 (e.g., MRI scanner and/or of fluoroscope 1015 and/or cone-beam CT scanner). In some embodiments, workstation 1001 may be coupled with the imaging device 1015, directly or indirectly, e.g., by wireless communication. Workstation 1001 may include a memory 1002, a processor 1004, a display 1006 and an input device 1010. Processor or hardware processor 1004 may include one or more hardware processors. Workstation 1001 may optionally include an output module 1012 and a network interface 1008. Memory 1002 may store an application 1018 and image data 1014. Application 1018 may include instructions executable by processor 1004 for executing the methods of the disclosure including the method of FIG. 4. As will be appreciated, the memory 1002 and the processor 1004 may be embodied in the cloud to offload local data storage and processing requirements.

Application 1018 may further include a user interface 1016. Image data 1014 may include the MRI data scans and 3D models derived from the MRI data scans and/or any other image data (e.g., from CT, fluoroscopy, or ultrasound) acquired of the patient either pre-procedurally or intra-procedurally (e.g., with a fluoroscope not shown). Processor 1004 may be coupled with memory 1002, display 1006, input device 1010, output module 1012, network interface 1008 and imaging device 1015. Workstation 1001 may be a stationary computing device, such as a personal computer, or a portable computing device such as a tablet computer. Workstation 1001 may embed a plurality of computer devices.

Memory 1002 may include any non-transitory computer-readable storage media for storing data and/or software including instructions that are executable by processor 1004 and which control the operation of workstation 1001 and, in some embodiments, may also control the operation of imaging device 1015. Imaging device 1015 may be used to capture a sequence of images based on which a 3D model is generated. In an embodiment, memory 1002 may include one or more storage devices such as solid-state storage devices, e.g., flash memory chips. Alternatively, or in addition to the one or more solid-state storage devices, memory 1002 may include one or more mass storage devices connected to the processor 1004 through a mass storage controller or a cloud or edge storage system (not shown) and a communications bus (not shown).

Although the description of computer-readable media contained herein refers to solid-state storage, it should be appreciated by those skilled in the art that computer-readable storage media can be any available media that can be accessed by the processor 1004. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by workstation 1001.

Application 1018 may, when executed by processor 1004, cause display 1006 to present user interface 1016. As will be appreciated any display may be employed including a monitor or screen, but also including an augmented reality, virtual reality, or extended reality headset. User interface 1016 may be configured to present to the user a screen including a three-dimensional (3D) view of a 3D model of a target from the perspective of a tip of a medical device. Additional views may also be displayed including a perspective view a distance further back from the distal end of the catheter 102 to show the distal tip and the target, an outside the airway view to show the catheter 102, airway, and the target. User interface 1016 may be further configured to display the target mark in different colors depending on whether the medical device tip is aligned with the target in three dimensions. This may be augmented with the display of one or more X, Y, Z vectors to illustrate the direction of any misalignment and the magnitude of the misalignment. Where a fluoroscope is employed, the user interface 1016 may display a live two-dimensional (2D) fluoroscopic view showing the navigation catheter and a target. Further, overlays and fusions of image data sets, regardless of when acquired or the modality may also be enabled (e.g., overlay or fusion of the 3D model of the target with/on the live 2D fluoroscopic image.) As will be appreciated, rotation of the fluoroscope while capturing the fluoroscopic images may be employed or required to generate a tomosynthesis and a 3D view using the fluoroscope.

Network interface 1008 may be configured to connect to a network such as a local area network (LAN) consisting of a wired network and/or a wireless network, a wide area network (WAN), a wireless mobile network, a Bluetooth network, and/or the Internet. As such, any of the hardware components of the system may employ either wired or wireless communications with other components of the system. Network interface 1008 may be used to connect between workstation 1001 and imaging device 1015. Network interface 1008 may be also used to receive image data 1014. Input device 1010 may be any device by which a user may interact with workstation 1001, such as, for example, a mouse, keyboard, foot pedal, touch screen, and/or voice interface. Output module 1012 may include any connectivity port or bus, such as, for example, parallel ports, serial ports, universal serial busses (USB), or any other similar connectivity port known to those skilled in the art.

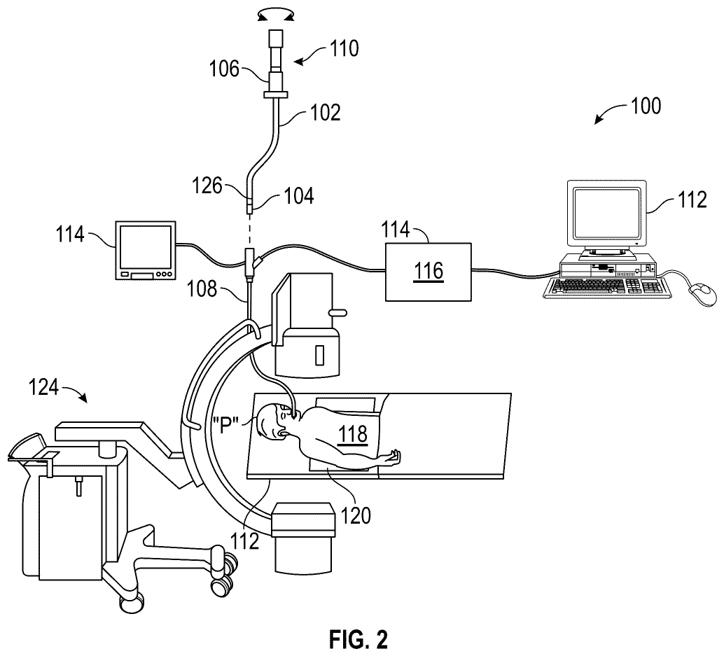

FIG. 2 depicts a perspective view of an exemplary system for facilitating navigation of a medical device, e.g., a catheter to a soft tissue target via airways of the lungs. System 100 may be further configured to construct fluoroscopic based three-dimensional volumetric data of the target area from 2D fluoroscopic images to confirm navigation to a desired location. System 100 may be further configured to facilitate approach of a medical device to the target area by using Electromagnetic Navigation (EMN) and for determining the location of a medical device with respect to the target. One such EMN system is the ILLUMISITE system currently sold by Medtronic PLC, though other systems for intraluminal navigation are considered within the scope of the disclosure including shape sensing technology which detect the shape of the distal portion of the catheter and match that shape to the shape of the luminal network in a 3D model.

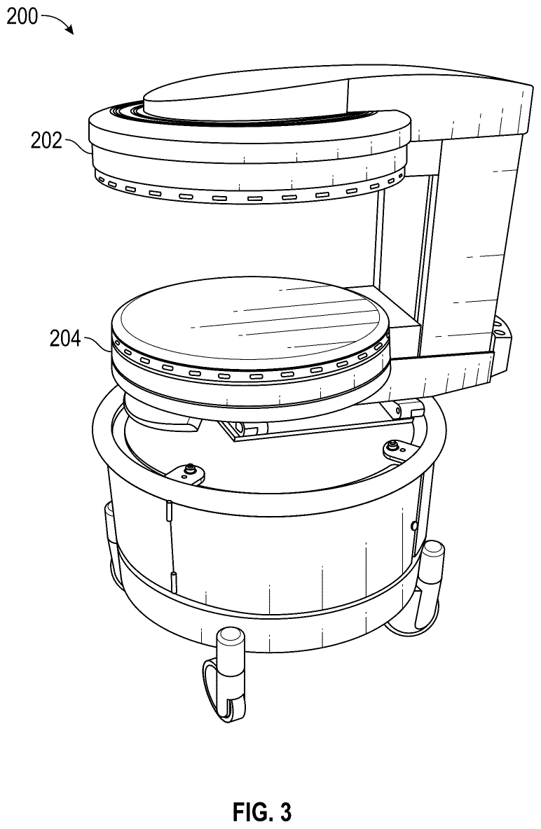

One aspect of the system 100 is a software component for reviewing of pre-procedure image scan data. Traditionally, the image scan data has been computed tomography (CT) image scan data that has been acquired separately from system 100. However, in accordance with one aspect of the disclosure, the image scan data may be MRI scan data such as might be received from a bed-side MRI device 200 as depicted in FIG. 3. The review of the image data allows a user to identify one or more targets, plan a pathway to an identified target (planning phase), navigate a catheter 102 to the target (navigation phase) using a user interface on computing device 122, and confirming placement of a sensor 104 relative to the target. The target may be tissue of interest identified by review of the image scan data during the planning phase. Following navigation, a medical device, such as a biopsy tool or a therapy tool (e.g., a microwave ablation tool, RF ablation tool, cyro ablation tool, during delivery tool, or another relevant tool) may be inserted into catheter 102 to obtain a tissue sample from the tissue located at, or proximate to, the target.

As shown in FIG. 2, catheter 102 is part of a catheter guide assembly 106. In one embodiment, catheter 102 is inserted into a bronchoscope 108 for access to a luminal network of the patient P. Specifically, catheter 102 of catheter guide assembly 106 may be inserted into a working channel of bronchoscope 108 for navigation through a patient's luminal network. The catheter 102 may itself include imaging capabilities and the bronchoscope 108 is not strictly required. A locatable guide (LG) 110 (a second catheter), including a sensor 104 may be inserted into catheter 102 and locked into position such that sensor 104 extends a desired distance beyond the distal tip of catheter 102. The position and orientation of sensor 104 relative to a reference coordinate system, and thus the distal portion of catheter 102, within an electromagnetic field can be derived, as described in greater detail below. Catheter guide assemblies 106 are currently marketed and sold by Medtronic PLC under the brand names SUPERDIMENSION® Procedure Kits, or EDGE™ Procedure Kits, and are contemplated as useable with the disclosure.

System 100 generally includes an operating table 112 configured to support a patient P, a bronchoscope 108 configured for insertion through patient P's mouth into patient P's airways; monitoring equipment 114 coupled to bronchoscope 108 or catheter 102 (e.g., a video display, for displaying the video images received from the video imaging system of bronchoscope 108 or the catheter 102); a locating or tracking system 114 including a locating module 116, a plurality of reference sensors 18 and a transmitter mat 120 including a plurality of incorporated markers; and a computing device 122 including software and/or hardware used to facilitate identification of a target, pathway planning to the target, navigation of a medical device to the target, and/or confirmation and/or determination of placement of catheter 102, or a suitable device therethrough, relative to the target.

In accordance with aspects of the disclosure, the visualization of intra-body navigation of a medical device, e.g., a biopsy tool, towards a target, e.g., a lesion, may be a portion of a larger workflow of a navigation system. Traditionally a fluoroscopic imaging device 124 capable of acquiring fluoroscopic or x-ray images or video of the patient P is also included in this particular aspect of system 100. The images, sequence of images, or video captured by fluoroscopic imaging device 124 may be stored within fluoroscopic imaging device 124 or transmitted to computing device 122 for storage, processing, and display. Additionally, fluoroscopic imaging device 124 may move relative to the patient P so that images may be acquired from different angles or perspectives relative to patient P to create a sequence of fluoroscopic images, such as a fluoroscopic video. The pose of fluoroscopic imaging device 124 relative to patient P while capturing the images may be estimated via markers incorporated with the transmitter mat 120. The markers are positioned under patient P, between patient P and operating table 112 and between patient P and a radiation source or a sensing unit of fluoroscopic imaging device 124. The markers incorporated with the transmitter mat 120 may be two separate elements which may be coupled in a fixed manner or alternatively may be manufactured as a single unit. Fluoroscopic imaging device 124 may include a single imaging device or more than one imaging device.

While the fluoroscopic imagining device 124 has traditionally been used for intraprocedural imaging and may still be employed in connection with aspects of the disclosure, an alternative imaging device such as the portable MRI scanner 200 depicted in FIG. 3 may also or alternatively be employed without departing from the scope of the disclosure. Because both the fluoroscopic imaging device 124 and the MRI scanner 200 are often portable, they may be used alternatively or in conjunction with one another without departing from the scope of the disclosure.

Computing device 122 may be any suitable computing device including a processor and storage medium, wherein the processor is capable of executing instructions stored on the storage medium. Computing device 122 may further include a database configured to store patient data, image data sets including CT image data sets (if any), MRI image data sets, fluoroscopic image data sets including 3D models derived from the image data sets, volumetric reconstructions, navigation plans, and any other such data. Although not explicitly illustrated, computing device 122 may include inputs, or may otherwise be configured to receive the image data sets described herein. Additionally, computing device 122 includes a display configured to display graphical user interfaces. Computing device 122 may be connected to one or more networks through which one or more databases may be accessed.

With respect to a planning phase, computing device 122 is configured with one or more applications that utilize an image data set to generate and enable viewing of a three-dimensional model or rendering of patient P's airways, enables the identification of a target on the three-dimensional model (automatically, semi-automatically, or manually), and allows for determining one or more pathways through patient P's airways to tissue located at and around the target. More specifically, the image data set may be processed and assembled into a three-dimensional volume, which is then utilized to generate a three-dimensional model of patient P's airways. The three-dimensional model may be displayed on a display associated with computing device 122, or in any other suitable fashion. Using computing device 122, various views of the three-dimensional model or enhanced two-dimensional images generated from the three-dimensional model are presented. The enhanced two-dimensional images may possess some three-dimensional capabilities because they are generated from three-dimensional data. The three-dimensional model may be manipulated to facilitate identification of target on the three-dimensional model or two-dimensional images, and selection of a suitable pathway through patient P's airways to access tissue located at the target can be made. As will be appreciated, a two-dimensional fluoroscopic image is a compression of an entire volume into a single plane. Thus, for each two-dimensional image depicted the orientation of the plane in the 3D volume may be displayed to provide information regarding the orientation of the fluoroscopic imaging device in relation to the 3D volume. Once selected, the pathway plan, three-dimensional model, and images derived therefrom, can be saved and exported, if required, to a navigation system for use during the navigation phase(s).

With respect to the navigation phase, one traditional method of determining the location of a catheter 102 employs a six degrees-of-freedom electromagnetic locating or tracking system 114, or other suitable system for determining position and orientation of a distal portion of the catheter 102. The tracking system 114 may, for example, be utilized for performing registration of the images and the pathway for navigation. Tracking system 114 includes the tracking module 116, a plurality of reference sensors 118, and the transmitter mat 120 (including the markers). Tracking system 114 is configured for use with a locatable guide 110 and particularly sensor 104. As described above, locatable guide 110 and sensor 104 are configured for insertion through catheter 102 into patient P's airways (either with or without bronchoscope 108) and are selectively lockable relative to one another via a locking mechanism.

A transmitter mat 120 is positioned beneath patient P. Transmitter mat 120 generates one or more electric or an electromagnetic fields around at least a portion of the patient P within which the position of a plurality of reference sensors 118 and the sensor 104 can be determined with use of a tracking module 116. One or more additional electromagnetic sensors 126 may also be incorporated into the end of the catheter 102. The second electromagnetic sensor 126 may be a five degree-of-freedom sensor or a six degree-of-freedom sensor. One or more of reference sensors 118 are attached to the chest of the patient P. Registration is generally necessary to coordinate locations of the three-dimensional model and two-dimensional images from the planning phase, with the patient P's airways as observed through the bronchoscope 108 and allow for the navigation phase to be undertaken with knowledge of the location of the sensor 104. Though, as discussed in further detail below, in embodiment where the pre-procedural imaging is acquired by the portable MRI device 200 and the patient P is not moved from operating table 112 where the images are acquired and the procedure will be undertaken, registration may not be required, or may only required at a later stage of the procedure following navigation proximate the target tissue.

Where registration is necessary, registration of the patient P's location on the transmitter mat 120 may be performed by moving sensor 104 through the airways of the patient P. More specifically, data pertaining to locations of sensor 104, while locatable guide 110 is moving through the airways, is recorded using transmitter mat 120, reference sensors 118, and tracking system 114. A shape resulting from this location data is compared to an interior geometry of passages of the three-dimensional model generated in the planning phase, and a location correlation between the shape and the three-dimensional model based on the comparison is determined, e.g., utilizing the software on computing device 122. In addition, the software identifies non-tissue space (e.g., air filled cavities) in the three-dimensional model. The software aligns, or registers, an image representing a location of sensor 104 with the three-dimensional model and/or two-dimensional images generated from the three-dimension model, which are based on the recorded location data and an assumption that locatable guide 110 remains located in non-tissue space in patient P's airways. Alternatively, a manual registration technique may be employed by navigating the bronchoscope 108 with the sensor 104 to locations in the lungs of the patient P, and manually correlating the images from the bronchoscope to the model data of the three-dimensional model. Additionally or alternatively, the distal tip of the catheter may be identified in the 3D volume and the 3D volume manually aligned to the pre-procedural images.

Though described herein with respect to EMN systems using EM sensors, the instant disclosure is not so limited and may be used in conjunction with flexible sensor, ultrasonic sensors, inertial measurement unit (sensors) or without sensors. Additionally, the methods described herein may be used in conjunction with robotic systems such that robotic actuators drive the catheter 102 or bronchoscope 108 proximate the target. These robotic systems may be autonomous, semi-autonomous, or nonautonomous systems that are motor driven, but surgeon controlled or supervised.

In one aspect of the disclosure a patient may be placed on the operating table 112 for imaging using the portable MRI scanner 200. The patient P is placed such that their chest is located between the upper magnetic coil 202 and the lower magnetic coil 204 of the electromagnet 206 of the MRI scanner 200. Application of a suitable radio frequency pulse signal to the upper magnetic coil 202 and the lower magnetic coil 204 generates a magnetic field of sufficient strength and homogeneity such that MR signals are emitted from any object placed in the magnetic field (e.g., at least a portion of the patient P such as the chest). In accordance with method 400, the emitted MR signals are captured and compiled to form an MRI image data set that is stored in the computing device 122 at step 402. As will be appreciated all MRI image data sets described herein may be captured with or without contrast agents. In one aspect of the disclosure, the image data set acquired by the MRI scanner 200 is the first image data set acquired for the procedure and is acquired at the time of the procedure. In accordance with this aspect, after acquisition of the MRI image data set, a 3D model is generated via one or more applications resident on the computing device 122 and presented on the display associated with the computing device 122 at step 404. As described above, the 3D model or the acquired MRI image data set may be analyzed and reviewed to identify target tissue and a pathway to the target tissue.

Though generally described in the context of MRI only imaging, the application is not so limited and those of skill in the art will understand that the method 400 may be similarly employed even if the 3D model generated at step 404 is in fact a 3D model generated from a CT scan. This 3D model may then require a registration step to register the 3D model to the patient, and enable accurate luminal navigation and fusing, overlaying, or updating of the relative position of the catheter 102 and the target(s) as described elsewhere herein. Once a target and a pathway are identified and planned in the MRI image data set, a catheter 102 may be inserted into the patient P and navigation to the target may be undertaken. As with other navigation systems the pathway through the airways to the target may be displayed on the display of the computing device 122 at step 406.

As described above, the catheter 102 may include one or more sensors 104,126. In accordance with the disclosure, one of the upper magnetic coil 202 or the lower magnetic coil 204 may be employed to generate an electromagnetic field at step 408 (e.g., in place of the transmitter mat 120). The sensors 104, 126 detect the electromagnetic field, which has been previously mapped to the MRI scanner 200 and to the surgical suite in which it is placed. The mapping of the electromagnetic field enables the signals received by the senor 104, 126 to be analyzed to determine the position of the sensor 104, 126 within the electromagnetic field at step 410.

The detected location of the sensor 104,126 in the electromagnetic field may then be displayed in the 3D model and/or 2D images acquired by the MRI scanner 200 using the display associated with the computing device 122 at step 412. Thus, as the catheter 102 is advanced along the pathway, the position of the sensor 104, 126 and the location of the distal portion of the catheter 102 can be updated and displayed in the 3D model on the display at step 414. As noted above, because the MRI scanner 200 is employed to the generate the 3D model, the coordinate systems of the MRI scanner 200 and particularly the electromagnetic field generated by the upper magnetic coil 202 and/or the lower magnetic coil 204 and the 3D model are necessarily and automatically registered to one another, thus there is no need to conduct any additional registration step that may be required when utilizing pre-procedural images acquired using a separate imaging device (e.g., a CT image scanner) that is located at a location outside the surgical suite.

As described above, a local registration has previously been employed to update the relative position of a distal portion of the catheter 102 and the target once the distal portion of the catheter is navigated to, for example, within about 3 cm of the target. The fluoroscope 124 may still be employed for this purpose without departing from the scope of the disclosure. However, as an alternative to the use of the fluoroscope 124, which employs x-rays and thus subjects the patient P and the surgical team to ionizing radiation, the MRI scanner 200 may be employed, though as also noted above a registration between the image data sets is not required when the same MRI scanner 200 is used without moving the patient P during the procedure.

In connection with use of the MRI scanner 200, a second MRI image data set may be acquired at step 416. This second MRI image data set is useful for assessing the relative locations of the catheter 102 and the target following navigation of the catheter 102 proximate the target. As is known, the tissues of the lungs have an elastic and flexible nature and thus because of the navigation of the catheter 102, the bronchoscope 108, or other tools through the locations and orientations airways of the patient the airways themselves can be distorted compared to their position prior to navigation. As a result, to ensure that a biopsy or therapy tool (e.g., a microwave ablation catheter) can be accurately placed in the target tissue the second MRI image data set depicts the lungs and airways in their potentially distorted positions.

To assist in resolution of the catheter 102, the catheter 102 may include one or more MRI visible markers may be incorporated into the catheter 102. The use of the MRI visible markers may be in addition to or as an alternative to the electromagnetic sensors 104,126. The MRI visible markers, either at the distal portion or along the length of the catheter 102 enable the clear identification of the catheter 102 in the second MRI image data set.

Following the acquisition of the second MRI image data set, the distal end of the catheter 102 and the target may again be identified and their relative position and orientation from one another calculated by one or more applications on the computing device 122 at step 418. This relative position and orientation can be used to generate and updated pathway to the target from the current location. Further, a second 3D model may also be generated from the second MRI image data, the second 3D model can be displayed on the display of the computing device, and the catheter 102 may be navigated the final few centimeters to the target. If desired, at step 420, a third MRI image data set may be acquired and reviewed in the display of the computing device 122 to ensure that the catheter 102 or a biopsy or therapy tool has been properly inserted into the target. Where the first MRI image dataset reveals multiple targets, following the conformation of placement of the biopsy or therapy tool and the acquisition of a biopsy or treatment of the target, and at least one target remains to be navigated to at step 422 the first 3D model may be again selected from the memory of the computing device 122 and displayed on the associated display at step 424 along with the pathways to the other targets, and the method reverts to step 410 and the process continues until all the targets have been navigated to and a biopsy or treatment applied.

As will be appreciated, during the acquisition of a biopsy or insertion of a therapy tool into a lesion or target, it may be desirable for the MRI scanner 200 to continuously capture MRI image data sets (e.g., a combination of steps 418 and 420) and continually display the updated relative positions of the catheter 102 and the target until the biopsy or therapy to is accurately placed within the lesion or target. This may be accompanied by the identification of the distal end of the catheter 102 (either with or without determining the catheter's trajectory) and the target at step 418, or the step 418 may be omitted.

Additionally or alternatively, it is contemplated herein that initial navigation of the catheter 102 may occur without any imaging (e.g., as a bronchoscope is) until arriving proximate a target. For example, this target may have been detected in some pre-procedural imaging. Following this navigation, the initial MRI image data set may be acquired to determine the relative position and orientation of the catheter 102 and the target. After the determination MRI image acquisition may be continuous till the diagnostic or therapy tool is inserted into the target tissue.

As noted above, the catheter 102 may include markers that are visible under MRI imaging. These markers enable the navigation of the catheter without the need for electromagnetic sensors 104, 126. This may be particularly useful where the there is no need to navigate to extreme peripheral sites within the lungs. Instead of utilizing an electromagnetic sensor 104, 126 and optical sensor (e.g., as typically employed on an endoscope or bronchoscope). Following the acquisition of the first MRI image data set at step 404 and generation of the first 3D model at step 406, a set of turn-by-turn directions is generated. These turn-by-turn directions enable the pulmonologist or the surgeon to navigate the catheter 102 to the target using the optical sensor. At each bifurcation in the airways when arrived at by the navigation of the catheter 102, the turn-by-turn directions provide an indication of which airway to proceed down. Again, as the catheter 102 approaches the target, or at any time when the pulmonologist or surgeon in unsure of which bifurcation to proceed down, a further MRI image data set may be captured with the MRI scanner 200. The markers that are visible in the MRI provide for an intraprocedural check of the location of either the distal portion of the catheter 102 or the entire length of the catheter 102 depending on the number of markers employed on the catheter 102. Each MRI image data set may output a revised turn-by turn direction set to guide the pulmonologist or surgeon to the destination and the steps may be repeated as many times as necessary until the catheter 102 arrives at the target. Further, each subsequent MRI data set may also supplant the previous 3D model displayed or may be used to correct registration of current 3D model displayed.

As will be appreciated utilizing the methods described above, the amount of ionizing radiation that the patient P and the surgical staff are exposed to is greatly reduced, and potentially completely eliminated. However, MRI scanners are not without their issues. As is known, high field strength devices (e.g., 1.5-3 tesla (T)) can cause injuries to the patient P when ferrous materials are in the presence of the generated field. However, it is believed that some low field strength MRI scanners (e.g., 0.2 T) and very low field strength MRI scanners (e.g., 0.1 T, 50 mT, and 20 mT) scanners can be operated without the potentially damaging heating effects.

Another aspect of MRI scanners is the time that is generally required to capture the MRI image data set. It can be common for a complete MRI image data set to require 30 min to even 1 hr. to capture the desired images. To address at least a portion of this issue, in accordance with the disclosure the scan volume of second and subsequent MRI image data sets can be reduced to just a volume around the distal portion of the catheter 102. Focusing the scan data to an area small enough to permit real-time scanning around the distal portion of the catheter 102 decreases the amount of time spent taking a scan. This has the added benefit that it should be less affected by patient motion due to the shorter scan time. In accordance with the embodiment described above without using electromagnetic navigation, the use of multiple sequential, potentially spatially adjacent or overlapping small volume scans at locations such as the distal portion of the catheter 102, the area proximate the lesion, and perhaps other locations selected by the pulmonologist or surgeon along the planned pathway. These may be taken at regular intervals, or for example following a certain number of the turn-by-turn navigation steps. By taking multiple scans at regular intervals the catheter 102 can be tracked visually in the MRI images as it traverses the patent's lungs until arriving at the target.

As a further alternative or additional option, the quality of the MRI scan data can be varied depending on the desired output of the scan. Where a large image volume is required but the overall quality is less important (e.g., for an initial scan, or for quickly confirming which branch of the airways to follow, the scan can be performed with a wider distance between slices of the slices of the entire volume. This enables the scans to be completed more quickly. Subsequently, as needed, the higher quality but smaller volume high resolution scans may also be acquired relatively quickly and the superimposed of fused with the lower quality MRI data set. Owing to the speed such small volume scans may be timed to minimize movement of the patient (e.g., to specific points during the cardiac cycle, or during portions of the ventilation cycle).

A further aspect of the differential in quality and scan time of the MRI data set can be achieved by using an initial very high scan quality MRI data set. As will be appreciated, this high-quality scan may take significant time, and may be acquired pre-procedurally or even at a different location from that of the surgical suite where the procedure may be undertaken. This high-quality MRI data set and the images derived from this MRI data set are the baseline for all future navigation of the patient. During the procedure, however, the MRI scanner can be set such that only a narrow field of view proximate the distal portion of the catheter 102 is acquired. This narrower field of view MRI data set may be of lesser quality that the original MRI image data set and employed and periodically or even continuously update the position of the distal portion of the catheter 102 within the patient. The data from the narrower field of view MRI image data set may or may not be fused with the original MRI data set. In once aspect on the position of the catheter 102 is determined and used to update the displayed position in a 3D model generated from the initial MRI image data set.

Though described above in conjunction with the upper magnetic coil 202 and/or the lower magnetic coil 204 generating the electromagnetic filed, the disclosure is not so limited. Rather in accordance with an aspect of the disclosure the transmitter mat 120 is placed between the upper magnetic coil 202 and the lower magnetic coil 204, for example below the patient and utilized to generate the electromagnetic field such that the sensor 104, 126 detects the location in the electromagnetic field. The transmitter mat may be energized during navigation and then de-energized any time an MRI image data scan is captured. In this way the electromagnetic fields do not interfere with one another. This may be achieved in the form of a software interlock where the MRI scanner 200 cannot be energized if the transmitter mat 120 is energized and vice versa.

Alternatively, rather than the transmitter mat 120 generating and transmitting a plurality of electromagnetic fields via multiple antennas the antennas can instead be receive antennas. The MRI scanner 200, as described above, generates an electromagnetic field. The electromagnetic fields can be detected by the receive antennas. As the catheter 102 is placed into the electromagnetic field generated by the MRI scanner 200, disturbances of the magnetic fields are also detected, and based on the location and magnitude of the disturbance the location of the catheter can be detected. The catheter 102 may be formed with a ferrous tip such that the disturbances caused by the ferrous tip are greater than other components of the catheter making the location of the distal tip more pronounced.

In a further aspect of the disclosure, instead of the transmitter mat 120 using discrete frequencies in the audible range that are transmitted through the antennas of the transmitter mat 120, a common carrier frequency on all emitting antennas can be employed. Each emitting antenna can be addressed with a specific digital signal. The digital signal may be as simple as a 3-bit binary signal (e.g., 001, 010, 011 . . . 110, 111) for a 9-antenna system. As will be understood by one of skill in the art a larger number of bit address may be needed in an array that has more antennas. The transmit signal could be complex with or without encryption to prevent reverse engineering.

The use of the common carrier frequency allows for more antenna elements in a distributed mesh network. Each element is uniquely addressed and allows for greater resolution of the electromagnetic field. The sensor 104, 126 receives a signal from a specific antenna pairing along with the adjacent antennas. The combination of the received signals from both the specific and adjacent antennas provides greater resolution of the specific location of the catheter element in the electromagnetic field. To ensure proper resolution of the signal, a parity bit or checksum can be employed to confirm receipt of the signal from a specific antenna in an effort to reduced impact of any induced error from external magnetic fields.

As noted above, one aspect of the disclosure involves the use of sensors within the catheter 102. One sensor type identified briefly above is an inertial measurement unit (IMU). An IMU is an electronic device that detects linear acceleration using one or more accelerometers, rotational rate using one or more gyroscopes, and one or more magnetometers for a heading reference. Often there is one accelerometer, one gyro, and one magnetometer per axis. These measurements from the IMU can be used by one or more software applications to calculate altitude, angular rates, linear velocity and position relative to a global reference. IMUs however are not perfect and are known to suffer from an accumulation of error which leads to drift (i.e., an ever-increasing error between where the software application believes the IMU is located and the actual location).

Within the context of use for this application, a catheter 102 utilizing an IMU can be used for following a pathway plan. With reference to the workflow described in FIG. 4, the process using an IMU includes placement of the catheter 102 within the field of view of the MRI scanner 200 prior to acquisition of the first MRI image data set, at step 402. The catheter 102 can be identified in the MRI image data set and provides an initial location of the IMU within the patient. As with method 400, a 3D model is generated and pathway through the 3D model to identified targets is formed (step 404) and displayed (step 406). At this point in the process, the process skips to step 412 where the detected position and orientation of the catheter 102 and particularly the IMU is updated on the display of the computing device 122, as the data from the included accelerometers, gyros, and magnetometers is received and analyzed by the one or more applications on the computing device 122. This data from the IMU is translated by the application to determine a change in location and orientation of the IMU from the location at the time of the acquisition of the MRI image data set from which the 3D model was generated. This change in orientation and location can then be displayed in the 3D model to provide an indication of the detected movement of the catheter 102 during the navigation through the patient. As noted above, the IMU suffers from drift over time. Accordingly, at periodic intervals (e.g., every few minutes) or at certain decision steps (e.g., bifurcations) or once within proximity of the target a second MRI image data set can be acquired (step 416) can be acquired. As described above, this may be a very narrow scope image data set focused just on the area around the distal portion of the catheter 102 (i.e., the IMU). With the second MRI image data set, the location of the distal portion of the catheter 102 can be updated in the 3D model. Further a new reference position for the IMU is generated eliminating the accumulated drift. As with the method 400 this process can be repeated many times until the catheter is placed in the target(s) and the biopsy and therapies applied.

As will be appreciated, the MRI image data set need to be gated to the cardiac or respiratory cycles. As such the new MRI image can be utilized used for locating the catheter 102 but the overall, the 3D MRI image may not be an exact replacement for a previous 3D MRI image due to things like chest expansion if taken on inhalation versus exhalation. Nonetheless, identification of the location of the catheter 102 and the relative position of the target remains quite useful to the user.

Alternatively, the catheter 102 may include one or more photoacoustic or ultrasound sensors to be used in conjunction with the IMU. These additional sensors can be placed proximate the distal end of the catheter 102. As the catheter 102 is advanced into the airways of the patient P, the photoacoustic or ultrasound sensors can provide image data from within the patient's airways. This data can be analyzed by one or more applications on the computing device 122 to determine the location of the catheter 102 and particularly the IMU within the airways of the patient. The determined position from the photoacoustic or ultrasound imaging may be used to update the data from the IMU to eliminate drift. The data in combination can be used then to update the depicted position of the distal portion of the catheter 102 in the 3D model generated from the first MRI image data scan.

Described above is the concept of image fusion. In image fusion, data from one image data set is utilized to update a portion of a second image data set. In one example, the image data sets received by the computing device 122 from the photoacoustic sensors or the ultrasound sensors, described above, are processed by one or more applications on the computing device and relevant portions fused with the first MRI image data set to update the image data set and provided for better differentiation of tissues, allowing for identification and differentiation of tissue types, or finding hidden details of the airways of the patient P that can only be ascertained by more local or more focused image modalities. Though described with reference to ultrasound and photoacoustic image sensors, the disclosure is not so limited and nearly any image data set may be fused wholly or in part with a second image data set. This includes prior pre-operative CT image data sets, fluoroscopic image data sets, MRI image data sets and others without departing from the scope of the disclosure.

As will be appreciated the fusion of image data sets, particularly those take at different times or in different locations will require registration of the image data sets. This may be accomplished by identifying similar structures in the two image data sets such that the coordinate system of the two image data sets can be oriented and aligned.

Relatedly, regarding imaging a further aspect of the disclosure employs the fluoroscope 124 for confirmatory purposes. The fluoroscope 124 has long been used to confirm placement of tools such as biopsy and therapy tools in the target tissue. In addition, because the fluoroscope 124 provides a “live image” the use of a fluoroscope 124 within the method 400, for example for the final navigation and insertion the catheter 102 or a biopsy or therapy tool into the target tissue. Still further, where therapy tools are concerned, the live imaging allows for the observation of the growth of an ablation zone as energy is applied to the therapy tool (e.g., an RF or microwave ablation catheter) inserted into the target. While the use of the fluoroscope 124 does result in the dosing of the patient and the surgical staff with ionizing radiation, in accordance with the disclosure through the use of MRI scanner 200 in combination with the fluoroscope 124, the net ionizing ration dose is still significantly reduced from current navigation, biopsy and therapy methods. As will be appreciated, the MRI scanner 200 and the fluoroscope 124 may be integrated into the operating table 112 and alternately deployed as needed. Alternatively, both imaging modalities may be portable such that they can be deployed and removed from the operating table 112 as needed. In some embodiments the MRI scanner is actually embedded in the operating table 112 and the fluoroscope 124 is supported by a robotic arm connected to the operating table.

Though described herein in conjunction with ablation therapies (e.g., RF or microwave ablation) the disclosure is not so limited, and a variety of therapies can be applied without departing from the scope of the disclosure. Obviously where RF or microwave ablations can be performed cryogenic ablations may also be performed by placing a cryo-ablation catheter within the target and releasing a cryogenic substance within the catheter such that energy is absorbed by the cryogenic substance to sub-cool the target and to kill the cells of the target. Alternatively, a variety of chemical ablations therapies have also been developed. Many of these are ethanol based, and when injected into the cells of the target denature and kill the cells to achieve the therapy.

As described above, the progression of therapy may be monitored in real-time using fluoroscope 124 as energy is applied to the target. To achieve a similar effect, the chemical ablation product may be enhanced with a contrast medium that is either visible in either MRI or fluoroscopic image data sets. Use of the contrast medium ensures that the therapy is applied to the target and further that the therapy remains in the target (e.g., within the capsule of a tumor) rather than drain away from the target via, for example, the lymphatic system.

Contrast medium are often used not just for enhancing the resolution of an injected tumor, as described above, but also to improve the images captured by the imaging modality for identifying different tissues. In accordance with a further aspect of the disclosure, because the imaging described herein is primarily of the lungs, cold air is employed as the contrast medium. During the acquisition cold air is circulated in conjunction with the ventilator through the patient's lungs. Based on the mechanism of the MRI scanner 200 in determining the boundaries of the tissues, the significant difference in temperature between the tissue of the lungs and the air circulating in the lungs. This cold air contrast medium enables the determination of boundaries of the airways in which the cold air is circulated and enhances the accuracy of the 3D model generated by the MRI scanner 200.

Returning to therapies useable with the systems of the disclosure, a further aspect of the disclosure is related to the modification of the catheter 102. Described above are modifications to make the catheter 102 more visible under MRI scanning through the use of markers either on the distal portion of the catheter 102 or along the length of the catheter 102. Similarly, the magnetic properties of the catheter 102. In one aspect of the disclosure, the magnetism of the catheter itself may be modified, (either increased or decreased) or the polarity of the magnetism of the catheter. These modifications of the magnetism can enable the manipulation of the catheter itself using the magnetic fields generated by the MRI scanner 200 itself. The magnetic fields generated by the MRI scanner 200 can be adjusted and manipulated to push and pull the catheter along the pathway to the target without necessarily requiring manual or robotic manipulation of the catheter.

Similar to manipulation of the magnetism of the catheter 102, the tip of the catheter 102 may be formed to include ferrous materials. Following placement of the catheter 102 into the target, the MRI scanner 200 may be energized. As is known the placement of a ferrous article in a magnetic field causes the ferrous article to heat. By adjusting the magnetic field generated by the MRI scanner 200, the amount of heat generated by the metal tip of the catheter can be controlled to deliver sufficient heat to the target such that the target tissue is denatured and the cells of the target (e.g., the tumor) are killed and the therapy achieved.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects.

We claim:

1. A system for luminal navigation:
a catheter configured for navigation within a luminal network of a patient, the catheter including a sensor; and
a computing device including a processor and computer readable memory, the memory storing thereon instructions that when executed by the processor:
receive magnetic resonance signals from a magnetic resonance image (MRI) scanner and to generate an MRI image data set;
generate a three-dimensional (3D) model from the MRI image data set;
generate a pathway through the 3D model to a target;
determine a location of the sensor within the patient;
cause display of a location of a portion of the catheter in the 3D model; and
update the displayed location of the portion of the catheter;
receive second magnetic resonance signals and generate a second MRI image data set;
receive an indication of a distal end of the catheter in the second MRI image data set; and
update a relative position of a distal end of the catheter and the target in the 3D model.

2. The system of claim 1, wherein the instructions when executed by the processor cause the display of the updated relative position of the distal end of the catheter and the target in the 3D model.

3. The system of claim 2, wherein the instructions when executed by the processor receive third magnetic resonance signals to form a third MRI image to confirm placement of the catheter, a biopsy tool, or a therapy tool in the target.

4. The system of claim 3, wherein the instructions when executed by the processor determine whether more targets exist in the 3D model.

5. The system of claim 4, wherein the instructions when executed by the processor cause the display of the 3D model and a pathway to a second target.

6. The system of claim 1, further comprising an magnetic resonance scanner generating the magnetic resonance signals.

7. The system of claim 1, wherein the instructions when executed by the processor cause the generation of an electromagnetic field and the sensor is an electromagnetic sensor.

8. The system of claim 7, further comprising a transmitter mat generating the electromagnetic field.

9. The system of claim 7, wherein a magnetic coil of the MRI scanner generates the electromagnetic field.

10. The system of claim 1, wherein the sensor is an inertial measurement unit.

11. A method of navigating a catheter to a target within a patient, the method comprising:
receiving magnetic resonance signals from a magnetic resonance image (MRI) scanner and generating an MRI image data set;
generating a three-dimensional (3D) model from the MRI image data set;
generating a pathway through the 3D model to a target;
determining a location of a sensor within the patient;
causing display of a location of a portion of a catheter in the 3D model based on the determined position of the sensor; and
updating a displayed location of the portion of the catheter;
receiving second magnetic resonance signals and generate a second MRI image data set;
receiving an indication of a distal end of the catheter in the second MRI image data set; and
updating a relative position of a distal end of the catheter and the target in the 3D model.

12. The method of claim 11, further comprising causing display of the updated relative position of the distal end of the catheter and the target in the 3D model.

13. The method of claim 12 further comprising receiving third magnetic resonance signals to form a third MRI image to confirm placement of the catheter, a biopsy tool, or a therapy tool in the target.

14. The method of claim 11, further comprising generating an electromagnetic field and the determining a location of the sensor in the electromagnetic field.

15. The method of claim 14, wherein the electromagnetic field is generated by a transmitter mat.

16. The method of claim 14, wherein a magnetic coil of the MRI scanner generates the electromagnetic field.

17. A method of navigating a catheter to a target within a patient, the method comprising:
receiving magnetic resonance signals from a magnetic resonance image (MRI) scanner and generating an MRI image data set;
generating a three-dimensional (3D) model from the MRI image data set;
generating a pathway through the 3D model to a target;
determining a location of a distal portion of a catheter within the 3D model;
causing display of the location of at least the distal portion of a catheter in the 3D model; and
receiving signals from a sensor incorporated in the catheter;
updating a displayed location of at least a portion of the catheter based on the received signals;
receiving second magnetic resonance signals and generate a second MRI image data set, wherein the second MRI image data set is focused to an area proximate the sensor; and
updating a displayed position of the distal portion of the catheter in the 3D model based on the second MRI image data set.

18. The method of claim 17, wherein the sensor is an inertial measurement unit (IMU).

19. The method of claim 18, wherein the updated displayed position of the distal portion of the catheter is employed to eliminate drift of the IMU.

20. The method of claim 19, further comprising receiving subsequent magnetic resonance signals and generating additional MRI image data sets, wherein the additional MRI image data sets are focused to the area proximate the sensor; and
updating the displayed position of the distal portion of the catheter in the 3D model based on the second MRI image data set, and
employing the updated displayed position of the catheter to eliminate drift of the IMU.
